(19) United States
(12) Patent Application Publication
(10) Pub. No.: US 2005/0033241 A1
(43) Pub. Date: Feb. 10, 2005

(54) Catheter protector

(76) Inventor: Molly Hottinger, St. Paul, MN (US)

Correspondence Address: DICKE, BILLIG & CZAJA, P.L.L.C., FIFTH STREET TOWERS, 100 SOUTH FIFTH STREET, SUITE 2250, MINNEAPOLIS, MN, 55402, US

(21) Appl. No.: 10/898,000

(22) Filed: Jul. 23, 2004

Related U.S. Application Data
(60) Provisional application No. 60/490,156, filed on Jul. 25, 2003.

(51) Int. Cl. A61M 5/32

(52) U.S. Cl. 604/179; 128/DIG. 6

(57) ABSTRACT

The present invention provides an improved catheter protector that comprises an elongated belt for encircling a patient's body and covering a catheter implant site. The protector allows ready and convenient access to an enclosed catheter and secures and protects the catheter during use while not damaging the patient's skin. The protector is comfortable to wear and may be constructed from washable and reusable fabrics or disposable materials. The catheter protector includes a pocket that is accessible from the edge of the belt and may be fabricated using inner and outer layers and a reclosable pocket fastener system for attaching the inner and outer layers together.

This application claims the benefit of U.S. Provisional Application No. 60/490,156, filed Jul. 25, 2003, which is incorporated herein in its entirety by reference.

### BACKGROUND

Tunneled central venous catheters are commonly used for prolonged treatment of cancer or other life-threatening diseases. The catheter is inserted in the chest of a patient (in close proximity of the central venous line) to administer medicated fluids into the blood distribution system or to perform periodic blood testing.

Historically, the exposed portion of the implanted catheter was taped to the skin of the patient near the entrance wound to prevent displacement and dislodgment of the catheter. Unfortunately, prolonged use of adhesive tapes to secure the catheter to the patient's skin causes skin irritation. Moreover, in the case of infant or child patients the patient is oftentimes able to pull on the exposed catheter, which may cause the catheter to dislodge from the vein, thus requiring a second surgical insertion.

Several attempts have been made to design catheter-retaining devices that overcome these disadvantages. These attempts have been unsuccessful. For example, certain prior art catheter retaining devices are designed such that the exposed portion of the catheter must be threaded through a hole in a band that wraps around the chest. This inconvenient design is disfavored and difficult to use. Other prior art devices require uncomfortable shoulder straps or don't adequately protect the catheter.

From the foregoing, it will be appreciated that what is needed in the art is a catheter protector that is suitable for young patients, cost efficient, comfortable, and easy to use while affording improved protection. Such a catheter protector is disclosed and claimed herein.

### SUMMARY

The present invention provides an improved catheter protector that comprises an elongated belt for encircling a patient's body and covering a catheter implant site. The protector allows ready and convenient access to an enclosed catheter and secures and protects the catheter during use while not damaging the patient's skin. The protector is comfortable to wear and may be constructed from washable and reusable fabrics or disposable materials.

The catheter protector belt includes an integral pocket having a reclosable pocket fastener system. The pocket is accessible from a region near one or two long edges of the belt. In a preferred embodiment the protector is fabricated using inner and outer layers of material and a reclosable pocket fastener system for attaching the inner and outer layers together.

One aspect of the present invention relates to a catheter protector including a belt for encircling a human torso, a pocket attached to the belt, and a reclosable fastener system. The pocket includes an outer surface, an inner surface adjacent to the belt, and an opening in the pocket for selective access to a catheter retaining space located between the outer surface and the inner surface. The reclosable fastener system is configured to secure the belt about the human torso. In one embodiment, the belt is a laminate comprising a first substantially inelastic nonwoven layer, an elastic layer, and a second substantially inelastic nonwoven layer. In this regard, the layers are cooperatively bonded together to form an elastic laminate.

### BRIEF DESCRIPTION OF THE DRAWINGS

FIG. 1 is a perspective view of a catheter protector including a belt according to one embodiment of the present invention.

FIG. 2 is a perspective view of a catheter protector attached about a torso according to one embodiment of the present invention.

FIG. 3 is a perspective view of an alternate catheter protector attached about a torso according to one embodiment of the present invention.

FIG. 4 is a perspective view of another alternate catheter protector attached about a torso according to one embodiment of the present invention.

FIG. 5 is a perspective view of yet another alternate catheter protector attached about a torso and including a pocket adjacent to a fastener according to one embodiment of the present invention.

FIG. 6 is a perspective view of still another alternate catheter protector attached about a torso and including a pocket defining a portion of a fastener system according to one embodiment of the present invention.

FIG. 7 is a cross-sectional view of a three-layer laminate belt according to one embodiment of the present invention.

FIG. 8 is a perspective view of an elastic layer of the three-layer laminate belt illustrated in FIG. 7.

### DETAILED DESCRIPTION

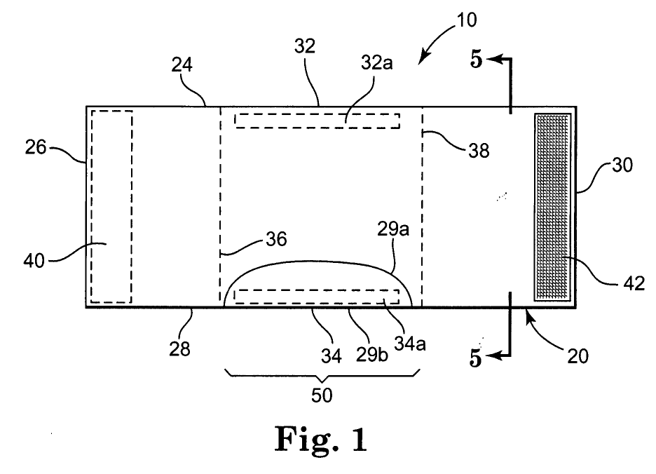

As shown in FIG. 1, the catheter protector 10 preferably comprises an elongated belt 20 for encircling a body and covering a catheter implant site. The belt may be constructed from a number of different materials. Suitable materials include woven or knit cloth as well as films and nonwoven materials. In one embodiment the belt is made of a reusable, washable fabric (e.g., cotton, polyester, rayon, blends of these fibers, etc.). In another embodiment the belt is made using disposable materials such as are used in the manufacture of disposable diapers (e.g., nonwoven materials). Preferred belts are made with comfortable latex-free materials, more preferably materials that are at least somewhat porous or “breathable.” The belt is also most preferably made with a material that is somewhat stretchy. This enables the belt to better conform to the patient's chest and to “move” as the chest expands during breathing.

The belt preferably comprises first and second ends (26, 30) and upper and lower edges (24, 28). At least the middle portion of the belt comprises inner and outer layers of material and defines a pocket portion 50. The pocket portion 50 comprises first and second seamed edges (36, 38) and an opening between the inner and outer layers along at least one of the upper and lower edges. FIG. 1 illustrates two such openings (32, 34), however, it is generally sufficient to have only one such opening. In the case of many fabrics first and second seamed edges will be sewn. In the case of non-woven materials the first and second seamed edges may comprise a weld line (e.g., a sonic weld line).

In one embodiment the pocket portion is formed using a patch of material that is roughly the size of the pocket. The patch is attached to the belt 20 along edges (36, 38). The pocket may have two openings (32, 34), or one of the openings may be shut and form a third edge. In an alternative embodiment, the belt 20 comprises two layers of material that run the length of the belt. The layers are attached at edges (36, 38) to form a pocket. The pocket may have one or two openings (32, 34) along the upper and lower edges (24, 28). The edges (36, 38) can be formed by folding material in half along a major axis and attaching the material together. In this case the protector would have only one opening (32 or 34).

A reclosable pocket fastener system (32a, 34a) is provided for securing the inner and outer layers of the pocket opening together and at least partially closing the pocket opening.

Suitable reclosable pocket fastener systems include securing systems that facilitate repeated open and closing of the pocket opening. In use, the belt covers the catheter implant site and the exposed end of the catheter is fitted into the pocket by opening the pocket at the edge of the belt. This permits entry to the pocket without having to remove the belt from the body.

Suitable reclosable pocket fastener systems include hook and loop closures (e.g., VELCRO-type systems), adhesive tapes, non-tacky fasteners, buttons, buckles, zippers, ties, magnets, hooks, etc. Preferred reclosable pocket fastener systems include hook and loop closures. The preferred reclosable pocket fastener systems allow ready access to the pocket so that the exposed segment of the catheter may be inserted into the pocket and secured therein. For many uses it is it is not necessary that the pocket fastening system (32a, 43a) completely close the opening. It is generally sufficient for the opening to be closed such that the catheter is retained therein during normal wearing. An advantage of VELCRO type fasteners is that they can close around the catheter. In preferred embodiments the catheter implant site is protected and not disturbed during routine insertion and removal from the pocket. Also in preferred embodiments the exposed end of the catheter may be readily accessed without needing to remove the catheter protector. This feature is particularly advantageous when the patient is an infant or young child and the catheter is being accessed while the patient is asleep.

The belt preferably comprises a reclosable fastener system that facilitates the catheter protector being wrapped and secured about the body to cover the catheter implant site. Suitable reclosable fastener systems can include the same type of systems as employed for the pocket opening. For example, the first and second ends (26, 30) of the belt may be fitted with hook and loop closures (e.g., VELCRO-type systems), adhesive tapes, non-tacky fasteners, buttons, buckles, zippers, ties, magnets, hooks, etc. Preferred reclosable fastener systems include hook and loop closures.

As illustrated in FIG. 1, the inner surface of the belt 20 has a fastener 40 attached near the first end 26. The outer surface of the belt 20 has a mating fastener 42 attached near the second end 30.

In one preferred embodiment the fastener 40 comprises a patch of “hook” material and the mating fastener 42 comprises a patch of “loop” material. When the belt is wrapped around the body and overlapped, the two fastener patches come in contact and secure the belt to the body. Alternatively, the outer surface of the-fabric belt near second end 30 can be selected of a material that can be gripped by a “hook” material.

In another alternative embodiment, the reclosable fastener system is made using a fastening system that comprises adhesive tape (e.g., where fastener 40 comprises a pressure sensitive adhesive patch (covered with a liner prior to use) and the outer surface of the belt near second end 30 is receptive to the adhesive patch or has a receiving patch secured thereto).

In yet another alternative embodiment, the reclosable fastener system comprises a “non-tacky fastener system” such as are described in U.S. Pat. Nos. 6,004,670; 5,912,670; 5,908,695; 5,888,335; 5,733,652; 5,316,849; and 5,196,266, which are herein incorporated by reference. Such non-tacky fastening systems comprise a non-tacky target surface and a contact responsive non-tacky fastening layer that adheres to the non-tacky target surface. Numerous polymeric materials may be used as the contact responsive fastening layer. The polymeric material may be a homopolymer, a random copolymer, a block copolymer, or a graft copolymer. It may be crosslinked or uncrosslinked. Specific examples of polymers useful as the fastening layer include ethylene-containing copolymers, urethane polymers such as urethanes prepared by the reaction of an isocyanate and an isocyanate-reactive compound, acrylic and acrylate polymers, urethane-acrylate polymers, butyl rubber, butadiene-acrylonitrile polymers, and butadiene-acylonitrile-isoprene polymers. Blends and mixtures of polymeric materials may be used if desired. Preferred polymers for use as the contact responsive fastening layer are selected from the group consisting of butadiene-acrylonitrile polymers, butadiene-acrylonitrile-isoprene polymers, urethane acrylate polymers, butyl rubber polymers, two-part urethane polymers, styrene-isoprene-styrene block copolymers and styrene-butadiene-styrene polymers.

Preferably the belt 20 covers and protects the implant area. More preferably, the entire catheter is secured by the belt and protected. This is especially desirable when the patient is an infant as unprotected catheters are susceptible of being grabbed by the infant or child causing damage or dislodgement.

In one preferred embodiment of the present invention the edge of the inner layer of the pocket near the opening (shown as 29a in FIG. 1) is set back from the edge of the outer layer near the opening 29b. With this design the entire catheter may be protected and covered by the belt. At the same time, the catheter does not require threading through an opening in the inner layer of the belt. This reduces the risk of pulling the catheter out of the body when removing the belt.

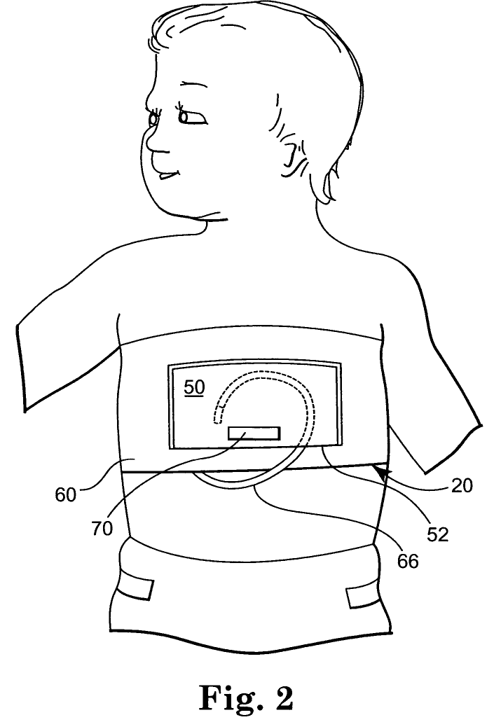

FIG. 2 illustrates a catheter protector according to one embodiment of the present invention secured to a child's body. In this embodiment the belt 20 comprises a band of a first material 60, a reclosable fastener system for closing the band around the body (not shown) and a pocket 50. Pocket 50 comprises a second piece of material that has been seamed on three sides. A fourth side 52 is openable. A catheter 66 is shown entering the pocket 50 and the pocket is closed using reclosable pocket fastener system 70. If desired, the open fourth side 52 could face upward (i.e., toward the head). The opening can be aligned with any of the edges, as dictated by convenience. In addition, if desired, the second piece of material may be attached on the inner surface of belt 20.

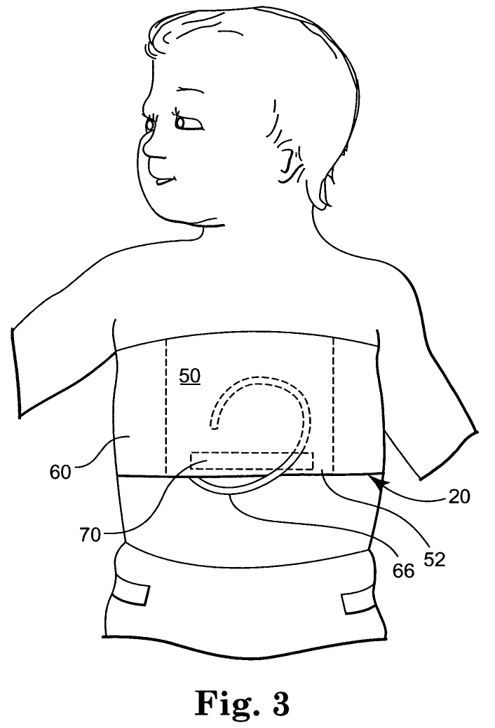

FIG. 3 illustrates a catheter protector according to one embodiment of the present invention secured to a child's body. In this embodiment the belt 20 comprises a two-layer band of material 60, a reclosable fastener system for closing the band around the body (not shown) and a pocket 50. Pocket 50 comprises a region of the band that has been closed about a periphery, leaving a fourth side 52 openable. A catheter 66 is shown entering the pocket 50 and the pocket is closed using reclosable pocket fastener system 70.

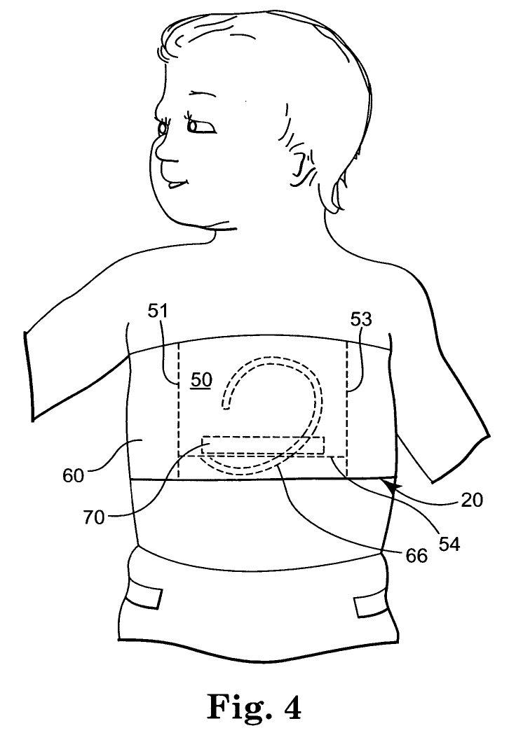

FIG. 4 illustrates a catheter protector according to one embodiment of the present invention secured to a child's body. In this embodiment the belt 20 comprises a two-layer band of material 60, a reclosable fastener system for closing the band around the body (not shown) and a pocket 50. Pocket 50 comprises a region of the band that has been closed on opposing sides 51 and 53. An access side 54 is openable. Notably, in this embodiment the lower edge of the inner layer of the pocket along the access side 54 is recessed from the edge of the band. This permits catheter 66 to enter the pocket 50 and be completely covered by the belt. The pocket 50 is closed using reclosable pocket fastener system 70.

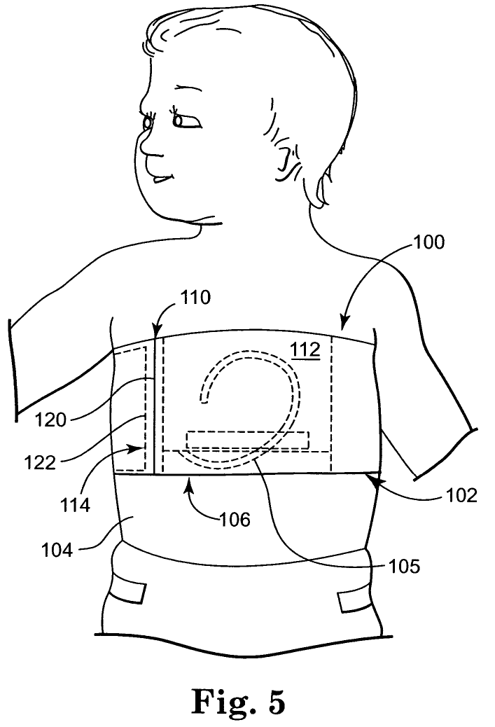

An alternate catheter protector 100 according to one embodiment of the present invention is illustrated in FIG. 5. The catheter protector 100 includes a belt 102 for encircling a body 104 and covering a catheter 105 at a catheter implant site 106. An end portion 110 of the belt 102 includes at least two layers of material overlapped to define a pocket 112 adapted to protectively cover the implant site 106. In this regard, a reclosable fastener 114 is configured to fasten adjacent to the end portion 110 such that manipulation of the fastener 114 is conducted adjacent to, and in view of, the pocket 112. In particular, the fastener 114 is shown having a tab end 120 secured over a target area 122. In one embodiment, the tab end 120 is a hook structure configured to reclosably fasten to a loop structure of the target area, although other fastener systems as described above are equally acceptable. In any regard, the catheter protector 100 can be removably fastened over the implant site 106 in a manner that permits a nurse or an infection prevention specialist to subsequently remove the belt 102 by removing the tab end 120 from the target area 122 while physically controlling movement of the catheter 105 at the implant site 106.

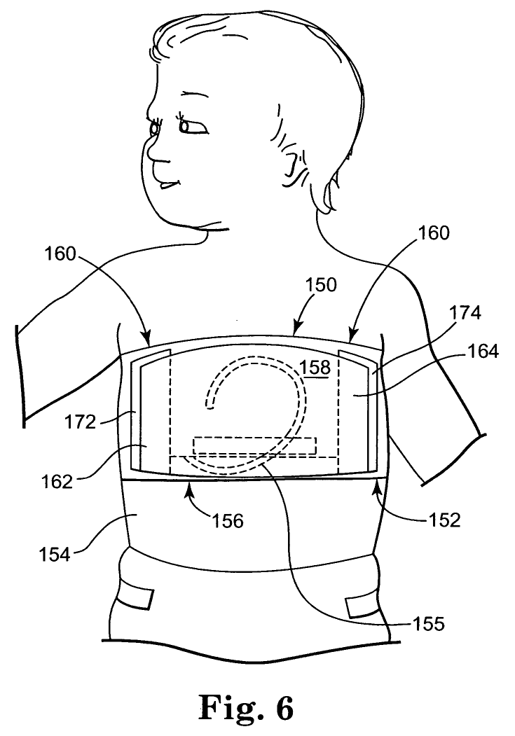

FIG. 6 is another embodiment of a catheter protector 150 according to the present invention. The catheter protector 150 includes a belt 152 for encircling a body 154 and covering a catheter 155 at a catheter implant site 156. A pocket 158 is provided that includes a fastener system 160. In one embodiment, the pocket 158 includes a first tab 162 and a second tab 164, the tabs 162, 164 configured to removably fasten to respective target areas 172 and 174. In one embodiment, the tabs 162, 164 include hook structures adapted to removably fasten to a loop structure formed by the target areas 172, 174, although other fastener systems as described above are equally acceptable. As illustrated in FIG. 6, the pocket 158 can be fastened to, and removed from, the belt 152, such that the pocket 158 defines a portion of the fastener system 160. In this manner, a nurse or an infection prevention specialist is afforded a clear view of the implant site 156 when removing or attaching the catheter protector 150.

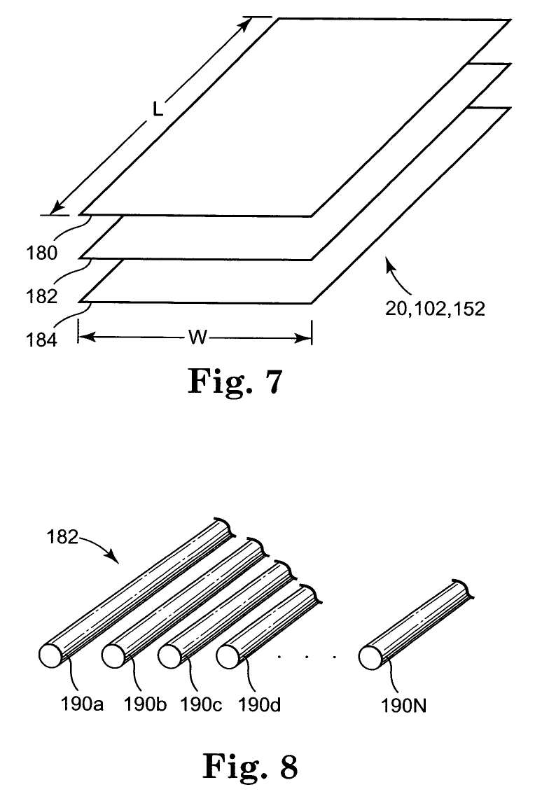

In one embodiment, the belts 20, 102, 152 shown above comprise a three-layer laminate suited for use as a catheter protector. In this regard, FIG. 7 is a cross-sectional view of one embodiment of the three-layer laminate belts 20, 102, 152. The belts 20, 102, 152 comprise a first substantially inelastic nonwoven layer 180, an elastic layer 182, and a second substantially inelastic nonwoven layer 184. The layers 180, 182, 184 are cooperatively bonded together to form an elastic laminate. In one embodiment, the elastic laminate has a machine direction oriented along a length L, where the length L is elastically deformable by at least 100 percent. In a preferred embodiment, the elastic laminate has a length L that is elastically deformable by at least 200 percent. In another embodiment, the belts 20, 102, 152 have a width W, where the width W is inelastically deformable.

As employed throughout this specification, elastically deformable is defined to be a recoverable elongation of the belts 20, 102, 152 to a second state that is longer than an initial length L1 of the belts 20, 102, 152. Recoverable elongation means that the belts 20, 102, 152 can be stretched from the initial length L1 to a second length L2, and upon relaxation, the elastically deformable belts 20, 102, 152 return to the initial length L1. That is to say, the belts 20, 102, 152 stretch from the initial length L1 to the second length L2 upon the application of a stretching force, and upon relaxation of the force, the belts 20, 102, 152 return to the initial length L1. In general, elastically deformable materials can be stretched by at least 50 percent and return, or recover, to the initial length L1. Conversely, inelastically deformable materials cannot be stretched more than approximately 10 percent without a permanent change in length. That is to say, an inelastically deformable material, when stretched from an initial length L1 to a second length L2, inelastically deforms such that the material is incapable of returning to the initial length L1.

Elastic layer 182 according to one embodiment of the present invention is illustrated in FIG. 8. The elastic layer 182 is comprised of discrete elastic strands 190. As illustrated in FIG. 8, a plurality of discrete elastic strands 190a, 190b. . . 190N are illustrated. Each of the elastic strands 190a. . . 190N is elastically deformable by at least 100 percent. In one embodiment, the elastic strands 190a. . . 190N are formed of an elastic polymeric material, such as polyurethane. The elastic strands 190a. . . 190N can be formed from other suitable elastic polymeric materials such as polyester, nylon, nylon 6, nylon 6,6, polybutylene, ethylene-containing copolymers, urethane-acrylate polymers, butadiene-acrylonitrile polymers, butadiene-acrylonitrile-isoprene polymers, blends and mixtures of said polymers, and copolymers. In one embodiment, the elastic strands 190a. . . 190N are SPANDEX brand elastic strands available from Du Pont, Wilmington, Del.

With reference to FIG. 7, the elastic layer 182 is disposed between the first and second substantially inelastic nonwoven layers 180, 184 such that an elastic laminate is formed that is suitable for use as the belts 20, 102, 152. For example, during formation, the elastic layer 182 is elongated by at least 100 percent and disposed between the inelastic layers 180, 184. In this elongated state, the elastic layer 182 is cooperatively bonded between the inelastic layers 180, 184 such that after bonding, the belts 20, 102, 152 are stretchy, i.e., elastically deformable. Specifically, as described above, the elastic laminate will have an elastic deformation of at least 100 percent. In a preferred embodiment, during formation, the elastic layer 182 is stretched by at least 200 percent prior to being cooperatively bonded to the inelastic layers 180, 184. In this preferred embodiment, the elastic laminate will have an elastic deformation of at least 200 percent.

With additional reference to FIG. 7, the substantially inelastic nonwoven layers 180, 184 are preferably made of polymeric materials. In a preferred embodiment, the substantially inelastic nonwoven layers 180, 184 are spun bonded nonwovens comprising polypropylene and formed as described in U.S. Pat. No. 5,814,349 (Geus et al.) incorporated herein by reference. After formation of the spun bonded nonwovens, the fibrous mat is point bonded by inserting the mat between calender rolls heated to approximately 300 degrees Fahrenheit and configured to exert approximately 40 psi pressure onto the mat.

With further reference to FIG. 7, the elastic layer 182 is preferably made of a nonwoven material. In one embodiment, the elastic layer 182 is a nonwoven material formed from a blown microfiber process as described in Wente, Van A., “Superfine Thermoplastic Fibers,” Industrial Engineering Chemistry, vol. 48, pp. 1342-46; in Report No. 4364 for the Naval Research Laboratories, published May 25, 1954, entitled, “Manufacture of Super Fine Organic Fibers” by Wente et al.; and in U.S. Pat. No. 4,215,682 (Kubik et al.) incorporated herein by reference. In another embodiment, the elastic layer 182 is a spun bonded nonwoven comprising an elastic polymer and formed as described in U.S. Pat. No. 5,814,349 (Geus et al.) incorporated herein by reference. In another embodiment, the elastic layer 182 is a nonwoven formed by a hybrid spun bond process as described in U.S. Pat. No. 6,607,624 (Berrigan et al.) incorporated herein by reference. In yet another embodiment, the elastic layer 182 is a nonwoven formed by a spunlace process as described in U.S. Pat. No. 4,612,237 (Frankenburg) incorporated herein by reference.

The pockets 50, 112, 158 can be attached to the three-layer laminate belts 20, 102, 152 shown in FIG. 1 and FIGS. 5-6 as described in any of the embodiments above. Specifically, the pocket 50 may have two openings (32, 34 as in FIG. 1), or one of the openings may be shut and form a third edge. That is to say, the pockets 50, 112, 158 can be formed of a second piece of material that is seamed on three of its sides, where the fourth side is openable for acceptance of the catheters 66, 106, 156.

Having thus described the preferred embodiments of the present invention, those of skill in the art will readily appreciate that the teachings found herein may be applied to yet other embodiments within the scope of the claims hereto attached.

1. A catheter protector, comprising:
a belt adapted for covering a catheter implant site, the belt including:
upper and lower edges,
a pocket defined by inner and outer layers, the pocket including first and second seamed edges and an opening between the inner and outer layers along at least one of the upper and lower edges to facilitate entry of a segment of an implanted catheter,
a reclosable pocket fastener system for attaching the inner and outer layers together and for securing the segment therein; and
a reclosable fastener system;
wherein the catheter protector encircles the body to cover the catheter implant site.

2. The protector of claim 1, wherein the belt comprises a latex-free cloth.

3. The protector of claim 1, wherein the belt comprises a nonwoven material.

4. The protector of claim 1, wherein the belt comprises a-stretchy material.

5. The protector of claim 1, wherein the belt comprises two layers of material.

6. The protector of claim 1, wherein the reclosable pocket fastener system is selected from the group consisting of hook and loop closures, adhesive tapes, non-tacky fasteners, buttons, buckles, zippers, ties, magnets, and hooks.

7. The protector of claim 1, wherein the reclosable fastener system is selected from the group consisting of hook and loop closures, adhesive tapes, non-tacky fasteners, buttons, buckles, zippers, ties, magnets, and hooks.

8. The protector of claim 1, wherein the belt is formed from two layers of material and the pocket is closed on three sides.

9. The protector of claim 1, wherein the inner edge of the pocket opening is recessed from the major edge of the belt.

10. A catheter protector, comprising:
an elongated belt for covering a catheter implant site, the belt formed of a nonwoven material and including:
upper and lower edges,
inner and outer layers, wherein a middle portion defines a pocket portion comprising first and second seamed edges and an opening between the inner and outer layers along one of the upper and lower edges to facilitate entry of an exposed segment of an implanted catheter,
a reclosable pocket fastener system for attaching the inner and outer layers together and securing the exposed segment therein; and
a reclosable fastener system for closing the protector around a body, wherein the catheter protector can be wrapped and secured about the body to cover the catheter implant site.

11. The protector of claim 10, wherein the inner edge of the pocket opening is recessed from the major edge of the belt.

12. A catheter protector comprising:
a belt for encircling a human torso;
a pocket attached to the belt, the pocket defining:
an outer surface, and
an inner surface adjacent to the belt, and
an opening in the pocket for selective access to a catheter retaining space located between the outer surface and the inner surface; and
a reclosable fastener system configured to secure the belt about the human torso;
wherein the belt is an elastic laminate comprising:
a first substantially inelastic nonwoven layer;
an elastic layer; and
a second substantially inelastic nonwoven layer, the layers cooperatively bonded together.

13. The catheter protector of claim 12, wherein the elastic laminate has a length and a width, the length elastically deformable by at least 100 percent.

14. The catheter protector of claim 12, wherein the elastic laminate has a length and a width, the length elastically deformable by at least 200 percent.

15. The catheter protector of claim 12, wherein the elastic laminate has a length and a width, the width being inelastically deformable.

16. The catheter protector of claim 12, wherein the elastic layer includes a plurality of discrete elastic strands.

17. The catheter protector of claim 16; wherein the discrete elastic strands are made of a material selected from the group consisting of polyester, nylon, nylon 6, nylon 6,6, polybutylene, ethylene-containing copolymers, urethane-acrylate polymers, butadiene-acrylonitrile polymers, butadiene-acrylonitrile-isoprene polymers, mixtures of polyester, mixtures of nylon, mixtures of nylon 6, mixtures of nylon 6,6, mixtures of polybutylene, mixtures of ethylene-containing copolymers, mixtures of urethane-acrylate polymers, mixtures of butadiene-acrylonitrile polymers, and mixtures of butadiene-acrylonitrile-isoprene polymers.

18. The catheter protector of claim 12, wherein the elastic layer includes an elastic nonwoven.

19. The catheter protector of claim 18, wherein the elastic nonwoven is formed from a process selected from the group consisting of blown microfiber, spunbond, hybrid spunbond, card, card-and-bond, and spunlace.

20. The catheter protector of claim 12, wherein at least one of the first and second substantially inelastic nonwoven layers is a spunbonded nonwoven.
